(19) United States
(12) Patent Application Publication
(10) Pub. No.: US 2010/0093678 A1
(43) Pub. Date: Apr. 15, 2010

(54) Compositions and methods of the treatment of obesity and osteoporosis

(75) Inventors: Mary Anne Della-Fera, Estes Park, CO (US); Srujana Rayalam, Athens, GA (US); Clifton A. Baile, Athens, GA (US)

Correspondence Address: COLEMAN SUDOL SAPONE, P.C., 714 COLORADO AVENUE, BRIDGE PORT, CT, 06605-1601, US

(73) Assignee: THE UNIVERSITY OF GEORGIA RESEARCH FOUNDATION, INC, Athens, GA (US)

(21) Appl. No.: 12/587,393

(22) Filed: Oct. 6, 2009

Related U.S. Application Data
(60) Provisional application No. 61/195,897, filed on Oct. 10, 2008.

(51) Int. Cl. A61K 31/59; A61P 3/04; A61P 19/10

(52) U.S. Cl. 514/167

(57) ABSTRACT

The present invention relates to naturally occurring compositions comprising vitamin D3 or related analog and methods for treating obesity and/or osteoporosis and reducing body fat or enhancing a bone graft in patients in need thereof.

### RELATED APPLICATIONS

This application claims the benefit of priority of U.S. provisional application Ser. No. 61/195,897, entitled “Compositions and Methods of the Treatment of Obesity and Osteoporosis, filed Oct. 10, 2008, the entire contents of which are incorporated by reference herein.

### FIELD OF THE INVENTION

The present invention relates to naturally occurring compositions and methods for treating obesity and/or osteoporosis and reducing body fat in patients in need thereof.

### BACKGROUND OF THE INVENTION

The hormonal metabolite of vitamin D, 1,25(OH)₂D₃, is best known for its important role in regulating levels of calcium and phosphorus in the body and in mineralization of bone. Appropriate concentrations of 1,25(OH)2D3 are required for optimal bone growth and the management of postmenopausal osteoporosis [1]. However, 1,25(OH)₂D₃ has been shown to act directly on both osteoblasts and on adipocytes [2]. The expression of adipocyte-specific transcription factors like C/EBPβ and PPARγ is markedly suppressed by 1,25(OH)₂D₃ in mouse epididymal fat tissue cultures [3] and 1,25(OH)₂D₃ induced apoptosis and inhibited adipogenesis in 3T3-L1 preadipocytes [3,4]. The antiproliferative effects of 1,25(OH)₂D₃ are reported to be mediated exclusively through the genomic signaling pathway by binding to a specific high affinity receptor protein, the 1,25-dihydroxyvitamin D3 receptor (VDR) [5]. VDR levels in adipocytes were shown to decline rapidly in the absence of 1,25(OH)₂D₃, whereas the presence of 1,25(OH)₂D₃ maintained VDR expression throughout the adipogenic program [6]. These and other recent studies suggest that vitamin D may play an important role in regulation of body fat content. For example, obese individuals were shown to have a greater risk for developing hyperparathyroidism, which is believed to be secondary to hypovitaminosis D [7]. Other studies have shown that circulating concentrations of vitamin D may be inversely related to the prevalence of diabetes and to blood glucose concentrations [8].

Guggulsterone (GS), a phytosterol isolated from the guggul tree Commiphora mukul, has been used in traditional medical practices to treat osteoarthritis and bone fractures. Interestingly, studies show that guggulsterone suppresses RANKL and tumor cell-induced osteoclastogenesis by suppressing the activation of NF-kappaB [9]. GS has also been found to reduce triglyceride and cholesterol levels and has been used to treat obesity [10]. Oral administration of GS was reported to decreases serum cholesterol levels in hypercholesterolemic rabbits [11], and GS decreased the body weight of humans and animals [12], suggesting that GS may directly affect adipocytes. Guggulsterone was recently shown to antagonize the farnesoid X receptor, a nuclear receptor that regulates gene expression in response to bile acid and an important regulator of cholesterol homeostasis [11].

The farnesoid X receptor (FXR) also plays a critical role in regulating adipogenesis and insulin signaling. During adipogenesis in 3T3-L1 cells, FXR gene expression rapidly increased in response to induction of differentiation and the expression peaked after 4 days of differentiation [12]. There is also evidence that VDR interacts directly with FXR. In a kidney cell model, VDR was shown to suppress the transactivation driven by chenodeoxycholic acid (a bile acid) interacting with FXR in a 1,25(OH)₂D₃-dependent manner [13]. This, in addition to the stabilization of VDR levels in adipocytes by 1,25(OH)₂D₃ leading to anti-adipogenic effects, suggested to us the possibility that the combination of 1,25(OH)₂D₃ and GS might lead to enhanced effects on adipocytes. Better understanding of the mechanisms through which dietary bioactives affect adipocyte size and number will help in developing treatments for prevention and progression of obesity and its associated diseases in humans. The objective of the study which gave rise to the present invention was to examine the possibility of interaction between 1,25(OH)₂D₃ and GS and determine the results of any interaction which might occur. Unexpectedly, this combination resulted in a synergistic and enhanced inhibition of adipogenesis and induction of apoptosis in maturing 3T3-L1 preadipocytes.

Aging is associated with detrimental changes in body composition, including loss of muscle mass (sarcopenia), loss of bone mass, and a relative increase in body fat. Even in the absence of obesity, elderly people can have a relative increase in body fat content, accompanied by an accumulation of adipocytes in non-adipose tissues such as muscle and bone marrow. Marrow adipocytes can inhibit osteoblast proliferation, stimulate the differentiation of osteoclasts, and disrupt the normal blood supply to bone tissue and bone forming cells. Treatments that inhibit marrow adipogenesis and reduce bone marrow adipocyte populations would therefore have significant, positive consequences for bone health. The present inventors have discovered that certain natural compounds, can be combined and act synergistically to promote osteogenesis, decrease adipogenesis and induce apoptosis of adipose tissue for treating conditions associated with increased adiposity and osteoporosis.

### BRIEF DESCRIPTION OF THE FIGURES

FIG. 1 shows the effect of GS+1,25(OH)2D3 on lipid content of maturing preadipocytes. (A) Lipid content was measured by Nile Red dye. Means not designated by a common superscript are different, ^{abcd}p<0.05. n=6; experiment repeated three times. (B) Representative images of Oil Red O staining to visualize intracellular triglyceride content.

FIG. 2 shows the effect of GS+1,25(OH)₂D₃ on maturing preadipocyte viability (A) and apoptosis (B) was quantified by ssDNA ELISA. Means not designated by a common superscript are different, ^{abcde}p<0.05. n=6; experiment repeated three times.

FIG. 3 shows the effect of Effect of GS+1,25(OH)₂D₃ on the expression of PPARγ, C/EBPα, and aP2. Means not designated by a common superscript are different, ^{abc}p<0.05.

FIG. 4 shows the effect of GS+1,25(OH)₂D₃ on VDR and FXR expression. (A) Effect of 1,25(OH)₂D₃ plus GS on VDR expression. (B) Effect of GS+1,25(OH)₂D₃ on FXR expression. Means not designated by a common superscript are different, ^{abc}p<0.05.

FIG. 5A-D show that both GS and XN reduced adipogenesis and promoted osteogenesis in hMSC cultured under adipogenic and osteogenic conditions, respectively. abcdef: Means that are not denoted with a common letter are different p<0.05.

FIG. 6 (A) shows pre-confluent human MSC which were treated with test compounds along with osteogenic induction media for 8 days. Alkaline phosphatase activity (ALP/protein) was measured on day 8. Means that are not denoted with a common letter are different (p<0.001). FIG. 6(B) shows human MSC which were treated with test compounds along with osteogenic induction media for 27 days and stained with Alizarin red S staining reagent. Cells treated with vitamin D (VD)+XN or VD+GS showed more calcium deposition than control and single compounds.

FIG. 7 shows that the combination of resveratrol (R)+quercetin (Q)+genistein (G) suppressed adipogenesis in Human MSC. Cells were cultured in adipogenic induction medium and treated with control or the combination of R+Q+G (15 μM each). After 18 days adipogenesis was measured and expressed as % control. Graph shows mean±SEM. Means that are not denoted with a common letter are different p<0.05.

FIG. 8 shows that quercetin reduced adipogenesis and promoted osteogenesis in hMSC cultured under adipogenic and osteogenic conditions, respectively. A. Lipid accumulation and B. Alp activity. (means that are not denoted with a common letter are different, abcde: p<0.05). C. Representative images of alizarin red staining. (abcde: p<0.05). D. Both genistein and 1,25(OH)₂D₃ significantly increased alp activity. * p<0.05; **p<0.01.

FIG. 9 shows the results of an in vivo experiment designed to determine the effectiveness of vitamin D+Resveretrol (R)+Quercetin (Q)+Genistein (G) in reducing adiposity and preventing bone loss in a rodent model of post-menopausal osteoporosis and weight gain. A. shows total body weight gain (g). B. shows the weight of retroperitoneal (R)+inguinal fat pads (g). C. shows the fat pad weight as a % of final body weight. Graphs show means±SEM a,b: means without a common letter are different, p<0.05.

FIG. 10 shows the results of the experiment briefly described in FIG. 9, above. Right femora were fixed for 24 hours in neutral buffered formalin and then stored in 70% ETOH prior to densitometry (PIXImus). Graphs show means±SEM. a,b: columns without a common letter are different, p<0.05.

### BRIEF DESCRIPTION OF THE INVENTION

The present invention relates to compositions comprising a combination of naturally occurring compounds and a vitamin D3 or a vitamin D3 analog, especially 1,25 dihydroxy vitamin D3, and their use to synergistically promote osteogenesis, decrease adipogenesis and increase apoptosis of adipose tissue. These compositions are useful for the treatment of osteoporosis and to reduce lipid accumulation and increase apoptosis of adipocytes, especially mature adipocytes, thus reducing body fat in a patient. Compositions and methods for treating osteoporosis and/or obesity represent aspects of the present invention. Further aspects of the invention include methods for reducing body fat, reducing body mass index and reducing visceral or intraabdominal fat or to enhance a bone graft in a patient or subject. In the case of enhancing a bone graft in a patient, it is believed that the compositions according to the present invention act by virtue of enhancing osteogenesis in a patient or subject administered the composition.

Methods according to the present invention comprise administering an effective amount of a vitamin D3 analog (especially 1,25 dihydroxy vitamin D3 or a compound which may readily form 1,25 dihydroxy vitamin D3 after administration) and at least one compound selected from the group consisting of guggulsterone, genistein, xanthohumol and mixtures thereof to treat conditions associated with increased adiposity and/or osteoporosis. Optionally, quercetin and/or resveratrol in effective amounts may also be included in compositions and methods according to the present invention. Thus, the present invention relates to compositions and methods for treating osteoporosis and/or to reduce body fat in a patient. In addition, the present invention relates to compositions according to the present invention which comprise an effective amount of xanthohumol and guggulsterone and optionally, one or both of a vitamin D3 analog (especially 1,25-dihydroxy vitamin D3) and/or genistein which are used for the treatment of osteoporosis, to reduce body fat in a patient or enhance a bone graft by enhancing osteogenesis as otherwise described herein. In further aspects of the invention, effective amounts of quercetin and/or reservatrol may be added to each of the above-described compositions and used in the methods of the present invention.

### DETAILED DESCRIPTION OF THE INVENTION

The following terms shall be used to describe the present invention. In instances where a term used to describe the present invention is not specifically defined herein, that term shall be given its traditional meaning when used, in context, by those of ordinary skill in the art.

As used herein and in the appended claims, the singular forms “a,” “an,” and “the” include plural reference unless the context clearly dictates otherwise. Thus, for example, a reference to “a compound” or other element of the present invention includes a plurality (for example, two or more elements) of such elements, and so forth. Under no circumstances is the patent to be interpreted to be limited to the specific examples or embodiments or methods specifically disclosed herein.

The term “compound”, as used herein, unless otherwise indicated, refers to any specific chemical compound disclosed herein and includes tautomers, regioisomers, geometric isomers, and where applicable, optical isomers thereof, as well as pharmaceutically acceptable salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds.

The term “patient” or “subject” is used throughout the specification within context to describe an animal, generally a mammal and preferably a human, to whom treatment, including prophylactic treatment, with the compositions according to the present invention is provided. For treatment of those infections, conditions or disease states which are specific for a specific animal such as a human patient, the term patient refers to that specific animal.

The term “effective” is used herein, unless otherwise indicated, to describe an amount (and length of therapy) of a compound or composition which, in context, is used to produce or effect an intended result, generally an amount which may be used to treat osteoporosis or obesity or reduce fat tissue in a patient in need of therapy or alternatively, is used to produce another compound, agent or composition. The term relates to an amount of a component used for a time period effective to produce an intended result. This term subsumes all other effective amount or effective concentration terms which are otherwise described in the present application.

The amount of Vitamin D (vitamin D3 or analog thereof) per day which is generally effective for use in the present invention ranges from about 25 μg to about 1.5 mg, about 50 μg to about 1.25 mg, about 100 μg to about 1.0 mg, about 100 μg to about 750 μg, about 100 to about 500 mg. In the case of genistein, when used, genistein is used in an amount per day ranging from about 5 to about 500 mg, about 10 to about 350 mg, about 15 to about 250 mg, about 25 to about 200 mg. In the case of guggulsterone, when used, guggulsterone is used in an amount per day ranging from about 5 mg to about 500 mg, about 10 to about 350 mg, about 15 to about 250 mg, about 25 to about 200 mg. In the case of xanthohumol, when used, xanthohumol is used in an amount per day ranging from about 500 μg to about 250 mg, about 1 mg to about 200 mg, about 2.5 to about 150 mg, about 5 to about 100 mg, about 5 to about 50 mg.

Quercetin and/or resveratrol may also be used in effective amounts in compositions according to the present invention. For quercetin the amount administered to a patient per day ranges from about 25 mg to about 2 g, about 100 mg to about 1.5 g, about 250 to about 1 g, about 500 mg to about 1 g. In the case of reseveratrol, the amount administered to a patient per day ranges from about 10 to about 750 mg, about 25 to about 650 mg, about 50 to about 600 mg, about 100 to about 500 mg., about 150 to about 400 mg.

As a guide without limitation, for ratios based upon weight, a vitamin D, genistein, xanthohumol combination may preferably range in weight based upon weight ratio for example, from about 1:100:25 to about 1:2000:1000, also about 1:250:50 to about 1:2000:500. A combination of vitamin D, guggulsterone and xanthohumol may preferably range in weight based upon weight ratio for example, from about 1:100:20 to about 1:2000:1000, also about 1:250:50 to about 1:2000:500. A combination of guggulsterone and xanthohumol may preferably range in weight from about 1:10 to about 10:1, about 5:1 to about 2:1. When all four compounds are used in combination, vitamin D, guggulsterone, xanthohumol and genistein may preferably range in weight, based upon weight ratio from about 1:100:25:100 to about 1:2000:1000:2000. In the case of quercetin and/or reservatrol, these compounds are added to the above-described formulations in amounts as other described hereinabove in effective amounts.

Each of the components may be administered as a bolus dose up to 4 (qid) or more times per day, generally once (in the case of sustained or controlled release compositions), twice (bid) or four times a day (qid). In addition, sustained release and/or controlled release versions of the compositions according to the present invention may also be used to administer compounds according to the present invention. The amount of each compound which is included in each composition to be administered will be a function of the total number of doses being given to a patient or subject each day, and the amount of each compound which is to be considered an effective amount, generally falling within the amounts and/or weight ratios which are described hereinabove.

The terms “vitamin D” or “vitamin D3 or an analog thereof” are used synonymously to refer to vitamin D3 compounds which find use in the present invention. The vitamin D3 analogs which find use in the present invention are those compounds, which include vitamin D3 (cholecalciferol), metabolites of vitamin D3, prodrug forms of cholecaliferol and related metabolites and their pharmaceutically acceptable salts, including 25-hydroxy vitamin D3 (calcidiol), which convert to 1,25-dihydroxy vitamin D3 (calcitriol), 1,25-dihydroxy vitamin D3 itself and prodrug forms. The preferred vitamin D3 analog which finds use in the present invention is 1,25-dihydroxy vitamin D3 (also known as 1α,25 dihydroxy vitamin D3 or calcitriol), which is a hormonal metabolite of vitamin D3. These terms refer to vitamin D3 and any analog or metabolite of vitamin D3 which produces or metabolizes into the vitamin D analog 1,25-dihydroxy vitamin D3, which is the active agent in the present invention. While 1,25-dihydroxy vitamin D3 is a preferred compound, any number of vitamin D3 analogs, metabolites and prodrug forms which are converted to 1,25-dihydroxy vitamin D3, as well as 1,25-dihydroxy vitamin D3 itself are useful in the present invention. In the body, 7-Dehydrocholesterol is the precursor of vitamin D₃ and forms cholecalciferol only after being exposed to solar UV radiation. Cholecalciferol is then hydroxylated in the liver to become calcidiol (25-hydroxyvitamin D₃). Next, calcidiol is again hydroxylated, this time in the kidney, and becomes calcitriol (1,25-dihydroxyvitamin D₃). Calcitriol is the most active hormone form of vitamin D₃.

The term “genistein” refers to one of several known isoflavones. Isoflavones, such as genistein, are found in a number of plants, with soybeans and soy products like tofu and textured vegetable protein being the primary food source. Genistein is a prooxidant flavonoid. Genistein is also known as 5,7-Dihydroxy-3-(4-hydroxyphenyl)chromen-4-one or 4′,5,7-Trihydroxyisoflavone. Soy isoflavones are a group of compounds found in and isolated from the soybean. The term genistein also refers to pharmaceutically acceptable salts thereof, where relevant.

The term “xanthohumol” refers to compound which is a prenylated chalcone, also a prenylflavonoid, and falls within the range of compounds called Xanthones (one of the primary compounds in St. Johns Wort). Xanthohumol was initially detected in an extract (series of Humulones) from Hops (Humulus lupulus), and is present in beer, although one would have to drink 120 gallons of beer per day to have any significant biological effect. Only comparatively minute quantities of xanthohumol are available in hops. Xanthohumol is obtainable in significant quantities from the Ashataba plant. Xanthohumol refers to a neutral compound and where relevant, a pharmaceutically acceptable salt thereof.

The term “guggulsterone” refers to a natural herb, which is a plant sterol found in and obtained from the resin (gum) of the guggul plant, Commiphora mukul. Molecules of guggulsterone have two chemical isomers E-guggulsterone and Z-Guggelsterone. Guggulsterone has few known side effects. It may be used “neat” as an isolated chemical entity in pure and crystallized E or Z form, as a mixture of stereoisomers, or as an extract obtained from gum (resin) guggul. Guggulsterone may be safely used in compositions according to the present invention.

The term “quercetin” refers to a plant-derived flavonoid, specifically a flavonol, used as a nutritional supplement. It has been shown to have anti-inflammatory and antioxidant properties and is being investigated for a wide range of potential health benefits. Quercetin is the aglycone form of a number of other flavonoid glycosides, such as rutin and quercitrin, found in citrus fruit, buckwheat and onions. Quercetin forms the glycosides quercitrin and rutin together with rhamnose and rutinose, respectively. Quercetin is classified in the IARC group 3 (no evidence of carcinogenicity in humans). Quercetin is also known by its IUPAC nomenclature as 2-(3,4-dihydroxyphenyl)-3,5,7-trihydroxy-4H-chromen-4-one. The structure of quercetin appears below.

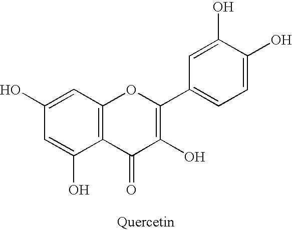

The term “resveratrol” refers to a phytoalexin produced naturally by several plants when under attack by pathogens such as bacteria or fungi. Resveratrol has also been produced by chemical synthesis and is sold as a nutritional supplement derived primarily from Japanese knotweed. Resveratrol is found in the skin of red grapes and is a constituent of red wine. Experiments have shown that resveratrol treatment extended the life of fruit flies, nematode worms and short living fish but it did not increase the life span of mice. Other names for resveratrol include trans-3,5,4′-Trihydroxystilbene; 3,4′,5-Stilbenetriol; trans-Resveratrol; and (E)-5-(p-Hydroxystyryl)resorcinol (E)-5-(4-hydroxystyryl)benzene-1,3-diol. The chemical structure of resveratrol appears below.

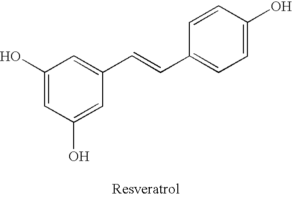

The term “pharmaceutically acceptable salt” refers to a salt (generally, but not exclusively an acid or base addition salt) of one or more of the compounds which may be used in the present invention. Thus, salts of vitamin D3 (especially metabolites or analogs of vitamin D3) where relevant, are contemplated for use in the present invention. The same is true for genistein, xanthohumol and guggelsterone, where relevant. The compounds used in the present invention are neutral when found in nature and are typically used in the present invention, but there pharmaceutically acceptable salts may be prepared for each.

The term “obesity” is used to describe a condition in which excess body fat has accumulated to such an extent that health may be negatively affected. It is commonly defined as a body mass index (BMI=weight divided by height squared) of 30 kg/m² or higher. This distinguishes it from being overweight as defined by a BMI of between 25-29.9 kg/m².

Excessive body weight is associated with various diseases, particularly cardiovascular diseases, diabetes mellitus type 2, insulin resistance, glucose intolerance, obstructive sleep apnea, certain types of cancer, and osteoarthritis. As a result, obesity has been found to reduce life expectancy. The primary treatment for obesity is dieting and physical exercise. If this fails, anti-obesity drugs and (in severe cases) bariatric surgery can be tried. Obesity arises from too much energy intake compared with a person's basal metabolic rate and level of physical exercise. Excessive caloric intake and a lack of physical activity in genetically susceptible individuals is thought to explain most cases of obesity, with purely genetic, medical, or psychiatric illness contributing to only a limited number of cases. With rates of adult and childhood obesity increasing, authorities view it as a serious public health problem.

“Body mass index” or BMI is a simple and widely used method for estimating body fat mass. BMI was developed in the 19th century by the Belgian statistician and anthropometrist Adolphe Quetelet. BMI is an accurate reflection of body fat percentage in the majority of the adult population, but is less accurate in situations that affect body composition such as in body builders and pregnancy.

BMI is calculated by dividing the subject's weight by the square of his/her height, typically expressed either in metric or US “Customary” units:

Metric: BMI=kg/m², where kg is the subject's weight in kilograms and m is the subject's height in metres. US/Customary and imperial: BMI=lb*703/in²
where lb is the subject's weight in pounds and in is the subject's height in inches. The most commonly used definitions, established by the WHO in 1997 and published in 2000, provide the following values:
A BMI less than 18.5 is underweight
A BMI of 18.5-24.9 is normal weight
A BMI of 25.0-29.9 is overweight
A BMI of 30.0-34.9 is class I obesity
A BMI of 35.0-39.9 is class II obesity
A BMI of >40.0 is class III obesity

Some modifications to the WHO definitions have been made by particular bodies:
A BMI of 35.0 or higher in the presence of at least one other significant comorbidity is also classified by some bodies as class III obesity.
For Asians, overweight is a BMI between 23 and 29.9 kg/m² and obesity a BMI >30 kg/m².

The surgical literature breaks down “class III” obesity into further catergories.
Any BMI >40 is severe obesity
A BMI of 40.0-49.9 is morbid obesity
A BMI of >50 is super obese

### Waist Circumference and Waist Hip Ratio

### Central Obesity

In those with a BMI under 35, intra-abdominal body fat is related to negative health outcomes independent of total body fat. Intra-abdominal or visceral fat has a particularly strong correlation with cardiovascular disease. In a study of 15,000 subjects, waist circumference also correlated better with metabolic syndrome than BMI. Women who have abdominal obesity have a cardiovascular risk similar to that of men. In people with a BMI over 35, measurement of waist circumference however adds little to the predictive power of BMI as most individuals with this BMI have an abnormal waist circumferences.

The absolute waist circumference (>102 cm in men and >88 cm in women) or waist-hip ratio (>0.9 for men and >0.85 for women) are both used as measures of central obesity.

The term “osteoporosis” is used to describe a disease or condition of bone that leads to an increased risk of fracture. In osteoporosis the bone mineral density (BMD) is reduced, bone micro-architecture is disrupted, and the amount and variety of non-collagenous proteins in bone is altered. Osteoporosis is defined by the World Health Organization (WHO) in women as a bone mineral density 2.5 standard deviations below peak bone mass (20-year-old healthy female average) as measured by DXA; the term “established osteoporosis” includes the presence of a fragility fracture.

Osteoporosis is most common in women after menopause, when it is called postmenopausal osteoporosis, but may also develop in men, and may occur in anyone in the presence of particular hormonal disorders and other chronic diseases or as a result of medications, specifically glucocorticoids, when the disease is called steroid- or glucocorticoid-induced osteoporosis (SIOP or GIOP). Given its influence on the risk of fragility fracture, osteoporosis may significantly affect life expectancy and quality of life. Osteoporosis can be prevented with lifestyle advice and sometimes medication, and in people with osteoporosis treatment may involve lifestyle advice, preventing falls and medication (calcium, vitamin D, bisphosphonates and several others).

### Signs and Symptoms

Osteoporosis itself has no specific symptoms; its main consequence is the increased risk of bone fractures. Osteoporotic fractures are those that occur in situations where healthy people would not normally break a bone; they are therefore regarded as fragility fractures. Typical fragility fractures occur in the vertebral column, rib, hip and wrist.

### Fractures

The symptoms of a vertebral collapse (“compression fracture”) are sudden back pain, often with radiculopathic pain (shooting pain due to nerve compression) and rarely with spinal cord compression or cauda equina syndrome. Multiple vertebral fractures lead to a stooped posture, loss of height, and chronic pain with resultant reduction in mobility.

Fractures of the long bones acutely impair mobility and may require surgery. Hip fracture, in particular, usually requires prompt surgery, as there are serious risks associated with a hip fracture, such as deep vein thrombosis and a pulmonary embolism, and increased mortality.

### Falls Risk

The increased risk of falling associated with aging leads to fractures of the wrist, spine and hip. The risk of falling, in turn, is increased by impaired eyesight due to any cause (e.g. glaucoma, macular degeneration), balance disorder, movement disorders (e.g. Parkinson's disease), dementia, and sarcopenia (age-related loss of skeletal muscle). Collapse (transient loss of postural tone with or without loss of consciousness) leads to a significant risk of falls; causes of syncope are manifold but may include cardiac arrhythmias (irregular heart beat), vasovagal syncope, orthostatic hypotension (abnormal drop in blood pressure on standing up) and seizures. Removal of obstacles and loose carpets in the living environment may substantially reduce falls. Those with previous falls, as well as those with a gait or balance disorder, are most at risk.

### Risk Factors

Risk factors for osteoporotic fracture can be split between non-modifiable and (potentially) modifiable. In addition, there are specific diseases and disorders in which osteoporosis is a recognized complication. Medication use is theoretically modifiable, although in many cases the use of medication that increases osteoporosis risk is unavoidable.

### Nonmodifiable Risks

The most important risk factors for osteoporosis are advanced age (in both men and women) and female sex; estrogen deficiency following menopause is correlated with a rapid reduction in BMD, while in men a decrease in testosterone levels has a comparable (but less pronounced) effect. While osteoporosis occurs in people from all ethnic groups, European or Asian ancestry predisposes for osteoporosis. Those with a family history of fracture or osteoporosis are at an increased risk; the heritability of the fracture as well as low bone mineral density are relatively high, ranging from 25 to 80 percent. There are at least 30 genes associated with the development of osteoporosis. Those who have already had a fracture are at least twice as likely to have another fracture compared to someone of the same age and sex.

### Potentially Modifiable Risks

Excess alcohol—small amounts of alcohol do not increase osteoporosis risk and may even be beneficial, but chronic heavy drinking (Alcohol intake greater than 2 units/day), especially at a younger age, increases risk significantly.

Vitamin D deficiency—low circulating Vitamin D is common among the elderly worldwide. Mild vitamin D insufficiency is associated with increased Parathyroid Hormone (PTH) production. ^{[10]}PTH increases bone reabsorption, leading to bone loss. A positive association exists between serum 1,25-dihydroxycholecalciferol levels and bone mineral density, while PTH is negatively associated with bone mineral density.

Tobacco smoking—tobacco smoking inhibits the activity of osteoblasts, and is an independent risk factor for osteoporosis. Smoking also results in increased breakdown of exogenous estrogen, lower body weight and earlier menopause, all of which contribute to lower bone mineral density.

High body mass index—being overweight protects against osteoporosis, either by increasing load or through the hormone leptin.

Malnutrition—low dietary calcium intake, low dietary intake of vitamins K and C Also low protein intake is associated with lower peak bone mass during adolescence and lower bone mineral density in elderly populations.

Physical inactivity—bone remodeling occurs in response to physical stress. Weight bearing exercise can increase peak bone mass achieved in adolescence. In adults, physical activity helps maintain bone mass, and can increase it by 1 or 2%. Conversely, physical inactivity can lead to significant bone loss.

Excess physical activity—excessive exercise can lead to constant damages to the bones which can cause exhaustion of the structures as described above. There are numerous examples of marathon runners who developed severe osteoporosis later in life. In women, heavy exercise can lead to decreased estrogen levels, which predisposes to osteoporosis. Intensive training is often associated with low body mass index.

Heavy metals—a strong association between cadmium, lead and bone disease has been established. Low level exposure to cadmium is associated with an increased loss of bone mineral density readily in both genders, leading to pain and increased risk of fractures, especially in the elderly and in females. Higher cadmium exposure results in osteomalacia (softening of the bone).

Soft drinks—some studies indicate that soft drinks (many of which contain phosphoric acid) may increase risk of osteoporosis; others suggest soft drinks may displace calcium-containing drinks from the diet rather than directly causing osteoporosis.

Diseases and Disorders Associated with Osteoporosis

Many diseases and disorders have been associated with osteoporosis. For some, the underlying mechanism influencing the bone metabolism is straight-forward, whereas for others the causes are multiple or unknown.

In general, immobilization causes bone loss (following the ‘use it or lose it’ rule). For example, localized osteoporosis can occur after prolonged immobilization of a fractured limb in a cast. This is also more common in active patients with a high bone turn-over (for example, athletes). Other examples include bone loss during space flight or in people who are bedridden or wheelchair-bound for various reasons.

Hypogonadal states can cause secondary osteoporosis. These include Turner syndrome, Klinefelter syndrome, Kallmann syndrome, anorexia nervosa, andropause, hypothalamic amenorrhea or hyperprolactinemia. In females, the effect of hypogonadism is mediated by estrogen deficiency. It can appear as early menopause (<45 years) or from prolonged premenopausal amenorrhea (>1 year). A bilateral oophorectomy (surgical removal of the ovaries) or a premature ovarian failure cause deficient estrogen production. In males, testosterone deficiency is the cause (for example, andropause or after surgical removal of the testes).

Endocrine disorders that can induce bone loss include Cushing's syndrome, hyperparathyroidism, thyrotoxicosis, hypothyroidism, diabetes mellitus type 1 and 2, acromegaly and adrenal insufficiency. In pregnancy and lactation, there can be a reversible bone loss.

Malnutrition, parenteral nutrition and malabsorption can lead to osteoporosis. Nutritional and gastrointestinal disorders that can predispose to osteoporosis include coeliac disease, Crohn's disease, lactose intolerance, surgery (after gastrectomy, intestinal bypass surgery or bowel resection) and severe liver disease (especially primary biliary cirrhosis). Patients with bulemia can also develop osteoporosis. Those with an otherwise adequate calcium intake can develop osteoporosis due to the inability to absorb calcium and/or vitamin D. Other micro-nutrients such as vitamin K or vitamin B12 deficiency may also contribute.

Patients with rheumatologic disorders like rheumatoid arthritis, ankylosing spondylitis, systemic lupus erythematosus and polyarticular juvenile idiopathic arthritis are at increased risk of osteoporosis, either as part of their disease or because of other risk factors (notably corticosteroid therapy). Systemic diseases such as amyloidosis and sarcoidosis can also lead to osteoporosis.

Renal insufficiency can lead to osteodystrophy.

Hematologic disorders linked to osteoporosis are multiple myeloma and other monoclonal gammopathies, lymphoma and leukemia, mastocytosis, hemophilia, sickle-cell disease and thalassemia.

Several inherited disorders have been linked to osteoporosis. These include osteogenesis imperfecta, Marfan syndrome, hemochromatosis, hypophosphatasia, glycogen storage diseases, homocystinuria, Ehlers-Danlos syndrome, porphyria, Menkes' syndrome, epidermolysis bullosa and Gaucher's disease.

People with scoliosis of unknown cause also have a higher risk of osteoporosis. Bone loss can be a feature of complex regional pain syndrome. It is also more frequent in people with Parkinson's disease and chronic obstructive pulmonary disease.

### Traditional Medication

Certain medications have been associated with an increase in osteoporosis risk; only steroids and anticonvulsants are classically associated, but evidence is emerging with regard to other drugs.

Steroid-induced osteoporosis (SIOP) arises due to use of glucocorticoids—analogous to Cushing's syndrome and involving mainly the axial skeleton. The synthetic glucocorticoid prescription drug prednisone is a main candidate after prolonged intake. Some professional guidelines recommend prophylaxis in patients who take the equivalent of more than 30 mg hydrocortisone (7.5 mg of prednisolone), especially when this is in excess of three months. Alternate day use may not prevent this complication.

Barbiturates, phenyloin and some other enzyme-inducing antiepileptics—these probably accelerate the metabolism of vitamin D.

L-Thyroxine over-replacement may contribute to osteoporosis, in a similar fashion as thyrotoxicosis does. This can be relevant in subclinical hypothyroidism.

Several drugs induce hypogonadism, for example aromatase inhibitors used in breast cancer, methotrexate and other anti-metabolite drugs, depot progesterone and gonadotropin-releasing hormone agonists.

Anticoagulants—long-term use of heparin is associated with a decrease in bone density, and warfarin (and related coumarins) have been linked with an increased risk in osteoporotic fracture in long-term use.

Proton pump inhibitors—these drugs inhibit the production of stomach acid; it is thought that this interferes with calcium absorption. Chronic phosphate binding may also occur with aluminium-containing antacids.

Thiazolidinediones (used for diabetes)—rosiglitazone and possibly pioglitazone, inhibitors of PPARγ, have been linked with an increased risk of osteoporosis and fracture.

Chronic lithium therapy has been associated with osteoporosis.

### Diagnosis

A scanner is used to measure bone density with dual energy X-ray absorptiometry. The diagnosis of osteoporosis is made on measuring the bone mineral density (BMD). The most popular method is dual energy X-ray absorptiometry (DXA or DEXA). In addition to the detection of abnormal BMD, the diagnosis of osteoporosis requires investigations into potentially modifiable underlying causes; this may be done with blood tests and X-rays. Depending on the likelihood of an underlying problem, investigations for cancer with metastasis to the bone, multiple myeloma, Cushing's disease and other above mentioned causes may be performed.

### Treatment

There are several alternatives of medication to treat osteoporosis, depending on gender, though lifestyle changes are also very frequently an aspect of treatment.

### Traditional Medication

Bisphosphonates are the main pharmacological measures for treatment. However, newer drugs have appeared in the 1990s, such as teriparatide and strontium ranelate.

### Bisphosphonates

In confirmed osteoporosis, bisphosphonate drugs are the first-line treatment in women. The most often prescribed bisphosphonates are presently sodium alendronate (Fosamax) 10 mg a day or 70 mg once a week, risedronate (Actonel) 5 mg a day or 35 mg once a week and or ibandronate (Boniva) once a month.

A 2007 manufacturer-supported study suggested that in patients who had suffered a low-impact hip fracture, annual infusion of 5 mg zoledronic acid reduced risk of any fracture by 35% (from 13.9 to 8.6%), vertebral fracture risk from 3.8% to 1.7% and non-vertebral fracture risk from 10.7% to 7.6%. This study also found a mortality benefit: after 1.9 years, 9.6% of the study group (as opposed to 13.3% of the control group) had died of any cause, indicating a mortality benefit of 28%.

Oral bisphosphonates are relatively poorly absorbed, and must therefore be taken on an empty stomach, with no food or drink to follow for the next 30 minutes. They are associated with esophagitis and are therefore sometimes poorly tolerated; weekly or monthly administration (depending on the preparation) decreases likelihood of esophagitis, and is now standard. Although intermittent dosing with the intravenous formulations such as zolendronate avoids oral tolerance problems, these agents are implicated at higher rates in a rare but unpleasant mouth disease called osteonecrosis of the jaw. For this reason, oral bisphosphonate therapy is probably to be preferred, and prescribing advice now recommends any remedial dental work to be carried out prior to commencing treatment.

### Teriparatide

Recently, teriparatide (Forteo, recombinant parathyroid hormone residues 1-34) has been shown to be effective in osteoporosis. It acts like parathyroid hormone and stimulates osteoblasts, thus increasing their activity. It is used mostly for patients with established osteoporosis (who have already fractured), have particularly low BMD or several risk factors for fracture or cannot tolerate the oral bisphosphonates. It is given as a daily injection with the use of a pen-type injection device. Teriparatide is only licensed for treatment if bisphosphonates have failed or are contraindicated (however, this differs by country and is not required by the FDA in the USA. However, patients with previous radiation therapy, or Paget's disease, or young patients should avoid this medication).

### Strontium Ranelate

Oral strontium ranelate is an alternative oral treatment, belonging to a class of drugs called “dual action bone agents” (DABAs) by its manufacturer. It has proven efficacy, especially in the prevention of vertebral fracture. In laboratory experiments, strontium ranelate was noted to stimulate the proliferation of osteoblasts, as well as inhibiting the proliferation of osteoclasts.

Strontium ranelate is taken as a 2 g oral suspension daily, and is licensed for the treatment of osteoporosis to prevent vertebral and hip fracture. Strontium ranelate has side effect benefits over the bisphosphonates, as it does not cause any form of upper GI side effect, which is the most common cause for medication withdrawal in osteoporosis. In studies a small increase in the risk of venous thromboembolism was noted, the cause for which has not been determined. This suggests it may be less suitable in patients at risk for thrombosis for different reasons. The uptake of (heavier) strontium in place of calcium into bone matrix results in a substantial and disproportionate increase in bone mineral density as measured on DXA scanning^{[36}], making further followup of bone density by this method harder to interpret for strontium treated patients. A correction algorithm has been devised.

Although strontium ranelate is effective, it's not approved for use in the United States yet. However, strontium citrate is available in the U.S. from several well-known vitamin manufacturers. Most researchers believe that strontium is safe and effective no matter what form it's used. Strontium, no matter what the form, must be water-soluble and ionized in the stomach acid. Stontium is then protein-bound for transport from the intestinal tract into the blood stream. Unlike drugs like sodium alendronate (Fosamax), strontium doesn't inhibit bone recycling and, in fact, may produce stronger bones. Studies have shown that after five years alendronate may even cause bone loss, while strontium continues to build bone during lifetime use. Strontium must not be taken with food or calcium-containing preparations as calcium competes with strontium during uptake. However, it's essential that calcium, magnesium, and vitamin D in therapeutic amounts must be taken daily, but not at the same time as strontium. Strontium should be taken on an empty stomach at night.

### Hormone Replacement

Estrogen replacement therapy remains a good treatment for prevention of osteoporosis but, at this time, is not recommended unless there are other indications for its use as well. There is uncertainty and controversy about whether estrogen should be recommended in women in the first decade after the menopause. In hypogonadal men testosterone has been shown to give improvement in bone quantity and quality, but, as of 2008, there are no studies of the effects on fractures or in men with a normal testosterone level.

Selective Estrogen Receptor Modulator (SERM)

SERMs are a class of medications that act on the estrogen receptors throughout the body in a selective manner. Normally, bone mineral density (BMD) is tightly regulated by a balance between osteoblast and osteoclast activity in the trabecular bone. Estrogen has a major role in regulation of the bone formation-resorption equilibrium, as it stimulates osteoblast activity. Some SERMs such as raloxifene (Evista), act on the bone by slowing bone resorption by the osteoclasts. Others, such as Femarelle (DT56a), achieve a significant effect by stimulating osteoblast activity thus inducing new bone formation, similarly to the estrogenic effect. Both have been proved as effective in clinical trials.

### Nutrition

Calcium

Calcium is required to support bone growth, bone healing and maintain bone strength and is one aspect of treatment for osteoporosis. Recommendations for calcium intake vary depending country and age; for individuals at higher risk of osteoporosis (after fifty years of age) the amount recommended by US health agencies is 1,200 mg per day. Calcium supplements can be used to increase dietary intake, and absorption is optimized through taking in several small (500 mg or less) doses throughout the day. The role of calcium in preventing and treating osteoporosis is unclear—some populations with extremely low calcium intake also have extremely low rates of bone fracture, and others with high rates of calcium intake through milk and milk products have higher rates of bone fracture. Other factors, such as protein, salt and vitamin D intake, exercise and exposure to sunlight, can all influence bone mineralization, making calcium intake one factor among many in the development of osteoporosis.

A meta-analysis of randomized controlled trials involving calcium and calcium plus vitamin D supported the use of high levels of calcium (1,200 mg or more) and vitamin D (800 IU or more), though outcomes varied depending on which measure was used to assess bone health (rates of fracture versus rates of bone loss). The meta-analysis, along with another study, also supported much better outcomes for patients with high compliance to the treatment protocol. In contrast, despite earlier reports in improved high density lipoprotein (HDL, “good cholesterol”) in calcium supplementation, a possible increase in the rate of myocardial infarction (heart attack) was found in a study in New Zealand in which 1471 women participated. If confirmed, this would indicate that calcium supplementation in women otherwise at low risk of fracture may cause more harm than good.

Vitamin D

Some studies have shown that a high intake of vitamin D reduces fractures in the elderly, though the Women's Health Initiative found that though calcium plus vitamin D did increase bone density, it did not affect hip fracture but did increase formation of kidney stones.

### Exercise

Multiple studies have shown that aerobics, weight bearing, and resistance exercises can all maintain or increase BMD in postmenopausal women. Many researchers have attempted to pinpoint which types of exercise are most effective at improving BMD and other metrics of bone quality, however results have varied. One year of regular jumping exercises appears to increase the BMD and moment of inertia of the proximal tibia in normal postmenopausal women. Treadmill walking, gymnastic training, stepping, jumping, endurance, and strength exercises all resulted in significant increases of L2-L4 BMD in osteopenic postmenopausal women. Strength training elicited improvements specifically in distal radius and hip BMD. Exercise combined with other pharmacological treatments such as hormone replacement therapy (HRT) has been shown to increases BMD more than HRT alone.

Additional benefits for osteoporotic patients other than BMD increase include improvements in balance, gait, and a reduction in risk of falls.

The term “bone grafting” refers to a surgical procedure that replaces missing bone with material from the patient's own body, an artificial, synthetic, or natural substitute. Bone grafting is used to repair bone fractures that are extremely complex, pose a significant risk to the patient, or fail to heal properly. Bone graft is also used to help fusion between vertebrae, correct deformities, or provide structural support for fractures of the spine. In addition to fracture repair, bone graft is used to repair defects in bone caused by birth defects, traumatic injury, or surgery for bone cancer.

Bone is composed of a matrix, mainly made up of a protein called collagen. It is strengthened by deposits of calcium and phosphate salts, called hydroxyapatite. Within and around this matrix are located the cells of the bones, which are of four types. Osteoblasts produce the bone matrix. Osteocytes are mature osteoblasts and serve to maintain the bone. Osteoclasts break down and remove bone tissue. Bone lining cells cover bone surfaces. Together, these four types of cells are responsible for building the bone matrix, maintaining it, and remodeling the bone as needed.

There are three ways in which a bone graft can help repair a defect. The first is called osteogenesis, the formation of new bone by the cells contained within the graft. The second is osteoinduction, a chemical process in which molecules contained within the graft enhance conversion of the patient's cells into cells that are capable of forming bone. The third is osteoconduction, a physical effect by which the matrix of the graft forms a scaffold on which cells in the recipient are able to form new bone.

New bone for grafting can be obtained from other bones in the patient's own body (e.g., hip bones or ribs), called autograft, or from bone taken from other people that is frozen and stored in tissue banks, called allograft. A variety of natural and synthetic replacement materials are also used instead of bone, including collagen (the protein substance of the white fibers of the skin, bone, and connective tissues); polymers, such as silicone and some acrylics; hydroxyapatite; calcium sulfate; and ceramics. Resorbable polymeric grafts are materials that provide a structure for new bone to grow on; the grafts then slowly dissolve, leaving only the new bone behind. Bone graft materials may also be enhanced by the addition of growth factors or morphogens that promote bone growth, such as bone morphogenic protein.

Compositions in this aspect of the invention include vitamin D3 or an analog thereof, in combination with at least one additional component selected from the group consisting of genistein, guggulsterone and xanthohumol, in combination with a carrier, additive or excipient (preferably, a bioresorbable or other polymeric material such as collagen matrix composition comprising collagen or other polymeric material which facilitates slow or sustained release of components from the composition at the site of the bone graft) all in effective amounts to enhance the likelihood (including synergistically) of a favorable bone graft.

The term “coadministration” refers to the administration of more than one active compound or component (e.g., vitamin D3 or an analog thereof, guggulsterone, genistein and/or xanthohumol) to a patient or subject which is used in the present invention at the same time such that the concentration of each compound in the blood, serum or plasma of the patient is maintained at effective levels. The term coadministration is not limited to the administration of more than compound at one time (at the same time), but rather to the administration of two or more compounds such that effective concentrations of each of the compounds is maintained, regardless of the time that a particular compound is administered. Thus, compounds according to the present invention may be administered over a broad range, including at or about at the same time. In preferred aspects of the invention, the compounds are administered at or about at the same time.

The present invention relates to compositions which comprise an effective amount of vitamin D3 or analog thereof (preferably 1,25-dihydroxy vitamin D or calcitriol) and guggulsterone and optionally, genistein and/or xanthohumol, or genistein and xanthohumol and optionally, vitamin D3 or analog thereof (preferably 1,25-dihydroxy vitamin D or calcitriol) and/or guggulsterone in combination with a pharmaceutically acceptable carrier, additive or excipient in treating osteoporosis, obesity or in reducing body fat, including visceral fat in a patient or subject and for use in bone graft materials to promote bone healing and bone growth. The compositions according to the present invention are shown to exhibit synergistic activity in treating osteoporosis, obesity and reducing body fat in a patient or subject. The present invention was not predictable expected from the available art.

The present invention also relates to methods of treating osteoporosis, obesity or reducing body fat in a patient or subject comprising administering to a patient or subject in need an effective amount of a composition which comprises an effective amount of vitamin D3 or analog thereof and guggulsterone and optionally, genistein and/or xanthohumol, or a composition which comprises an effective amount of genistein and xanthohumol and optionally, vitamin D3 or analog thereof and/or guggulsterone optionally in combination with a pharmaceutically acceptable carrier, additive or excipient.

The present invention also relates to methods to promoting bone growth in bone defects by the inclusion in bone grafting materials of effective amounts of a composition which comprises an effective amount of vitamin D3 or analog thereof and guggulsterone and optionally, genistein and/or xanthohumol, or a composition which comprises an effective amount of genistein and xanthohumol and optionally, vitamin D3 or analog thereof and/or guggulsterone.

The present invention relates to compositions and methods for the treatment of osteoporosis and/or obesity, as well as reducing body fat in a patient or subject. Methods of reducing visceral or intra-abdominal fat (fat mass) are also aspects of the present invention. In particular, the present inventors have demonstrated the activity and the molecular mechanisms responsible for the synergistic effects of combinations of specific natural compounds with vitamin D3 or an analog thereof that forms the basis for the present invention. The present inventors have found that combinations of 1,25 dihydroxy vitamin D3 and guggulsterone, 1,25 or dihydroxy vitamin D3+genistein+xanthohumol synergistically and dramatically reduced lipid accumulation and increased apoptosis in preadipocytes and induce apoptosis of mature adipocytes. This enhanced activity was unexpected because it occurred at concentrations that had little to no effect when the compounds were tested individually. We have also shown that xanthohumol and guggulsterone inhibit adipogenesis and promote osteogenesis in bone marrow stem cells. Vitamin D3 or an analog thereof in various combinations of genistein, guggulsterone and xanthohumol or botanical extracts or isolates containing these compounds in appropriate amounts and proportions could therefore be used as effective treatments for obesity and osteoporosis in a patient or subject.

Compounds used in the present invention may be used in pharmaceutical compositions having biological/pharmacological activity for the treatment of osteoporosis or obesity, or to reduce body fat, including visceral fat in a patient or subject, or both. These compositions comprise an effective amount of any one or more of the compounds disclosed hereinabove, optionally in combination with a pharmaceutically acceptable additive, carrier or excipient. Compounds according to the present invention may also be used as intermediates in the synthesis of compounds exhibiting biological activity as well as standards for determining the biological activity of the present compounds as well as other biologically active compounds.

The compositions of the present invention may be formulated in a conventional manner using one or more pharmaceutically acceptable carriers. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, sublingually, buccally, vaginally, by inclusion in bone grafting materials or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally, or intravenously. Preferred routes of administration include oral administration, sublingual or buccal administration (quick release and/or sustained/controlled release).

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Hely or similar alcohol.

The compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The compositions of this invention may also be administered topically, especially when the target of treatment includes areas or organs readily accessible by topical application. Suitable topical formulations are readily prepared for each of these areas or organs.

Topical application also can be effected in a rectal suppository formulation (see above) or in a suitable enema formulation. Topically-transdermal patches may also be used.

For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The compositions of this invention may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compositions of this invention may also be included in any suitable bone graft material, such as autologous and non-autologous bone materials and synthetic bone grafting materials.

The amount of compound of the instant invention that may be combined with the carrier materials to produce a single dosage form will vary depending upon the host treated, the particular mode of administration. Preferably, the compositions should be formulated so that a therapeutically effective dosage of between about 0.05 and 25 mg/kg, about 2.5 to about 20 mg/kg about 5 to about 15 mg/kg of patient/day of the active compounds can be administered to a patient receiving these compositions. Preferably, compositions in dosage form according to the present invention comprise a therapeutically effective amount of at least 1 mg of active compound, at least 2.5 mg of active compound, at least 5 mg of active compound, at least 10 mg of active compound, at least 15 mg of active compound, at least 25 mg of active compound, at least 50 mg of active compound, at least 60 mg of active compound, at least 75 mg of active compound, at least 100 mg of active compound, at least 150 mg of active compound, at least 200 mg of active compound, at least 250 mg of active compound, at least 300 mg of active compound, about 350 mg of active compound, about 400 mg of active compound, about 500 mg of active compound, about 750 mg of active compound, about 1 g (1000 mg) of active compound. It is noted that each of the active compounds used in the compositions according to the present invention may be used in varying amounts, within the above descriptive limits.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Administration of the active compound may range from continuous (intravenous drip) to several oral or inhalation (intratracheal) administrations per day (for example, B.I.D. or Q.I.D.) and may include oral, pulmonary, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Oral, buccal and sublingual dosage forms are particularly preferred, because of ease of administration and prospective favorable patient compliance.

To prepare the compositions according to the present invention, a therapeutically effective amount of a combination of compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral, buccal, sublingual or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques. The use of these dosage forms may significantly the bioavailability of the compounds in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of free nucleosides, acyl/alkyl nucleosides or phosphate ester pro-drug forms of the nucleoside compounds according to the present invention.

The present invention also preferably relates to compositions in oral dosage form comprising therapeutically effective amounts of active compound according to the present invention, optionally in combination with a pharmaceutically acceptable carrier, additive or excipient. Compositions for oral administration include powders or granules, suspensions or solutions in water or non-aqueous media, sachets, capsules or tablets. Thickeners, diluents, flavorings, dispersing aids, emulsifiers or binders may be desirable.

The pharmaceutical compositions of the invention are safe and effective for use in the therapeutic methods according to the present invention. Although the dosage of the composition of the invention may vary depending on the type of active substance administered (vitamin D3 or analog thereof, genistein, xanthohumol, guggulsterone and optional agents, where relevant) as well as the nature (size, weight, etc.) of the subject to be diagnosed, the composition is administered in an amount effective for allowing the pharmacologically active substance to be cleaved to cleavage products to be measured. For example, the composition is preferably administered such that the active ingredients (active compound) can be given to a human adult in a dose of at least about 1 mg, at least about 2.5 mg, at least about 5 mg, at least about 10 mg, at least about 15 mg, at least about 20 mg, at least about 25 mg, at least about 50 mg, at least about 60 mg, at least about 75 mg., at least about 100 mg, at least about 150 mg, at least about 200 mg, at least about 250 mg, at least about 300 mg, at least about 350 mg, at least about 400 mg, at least about 500 mg, at least about 750 mg, at least about 1000 mg, and given in a single dose, including sustained or controlled release dosages once daily.

The form of the pharmaceutical composition of the invention such as a powder, solution, suspension etc. may be suitably selected according to the type of substance to be administered.

As an administration route, direct inhalation via the mouth using an inhaler is usually administered into the airways and in particular, directly to pulmonary tissue, the active substance contained therein produces immediate effects. Furthermore, the composition is formulated as an immediate release product so that cleavage and analysis can begin soon after administration.

### EXAMPLES

### Methods and Procedures

### Reagents.

Phosphate-buffered saline (PBS) and Dulbecco's modified Eagle's medium (DMEM) were purchased from Gibco (BRL Life Technologies, Grand Island, N.Y.). ApoStrand ELISA Apoptosis Detection Kits and 1,25(OH)2D3 were purchased from BIOMOL (Plymouth Meeting, Pa.). The viability assay kit (CellTiter 96 Aqueous one solution cell proliferation assay) was purchased from Promega (Madison, Wis.). Oil Red O stain and Hoechst stain were from Sigma (St. Louis, Mo.) and AdipoRed™ Assay Reagent was from Cambrex BioScience Walkersville, Inc. (Walkersville, Mass.). cis-Guggulsterone was purchased from Steraloids, Inc. (Newport, R.I.). Antibodies specific for b-actin, C/EBP beta, aP2, PPAR gamma, and FXR were from Santa Cruz Biotechnology (Santa Cruz, Calif.). Anti-Vitamin D Receptor antibody was purchased from Affinity Bioreagents (Golden, Colo.) and all the secondary antibodies were from Santa Cruz Biotechnology (Santa Cruz, Calif.).

### Cell Line and Cell Culture.

3T3-L1 mouse embryo fibroblasts were obtained from American Type Culture Collection (Manassas, Va.) and were cultured as described elsewhere [14]. Briefly, cells were cultured in DMEM containing 10% bovine calf serum until confluent. Two days after confluence, the cells were stimulated to differentiate with DMEM containing 10% fetal bovine serum (FBS), 167 nM insulin, 0.5 μM isobutylmethylxanthine (MMX), and 1 μM dexamethasone for 2 days. On day 2, differentiation medium was replaced with 10% FBS/DMEM containing 167 nM insulin and incubated for 2 days, followed by culturing with 10% FBS/DMEM for an additional 4 days, at which time >90% of cells were mature adipocytes with accumulated fat droplets. All media contained 1% penicillin streptomycin (10,000 U/ml) and 1% (v/v) 100 mM pyruvate. Cells were maintained at 37° C. in a humidified 5% CO2 atmosphere.

### Quantification of Lipid Content

Lipid content was quantified using commercially available AdipoRed™ Assay Reagent. In brief, maturing adipocytes grown in 96-well plates were incubated with vehicle or test compounds during the adipogenic phase and on day 6, culture supernatant was removed and lipid content was quantified by performing AdipoRed™ assay as per the manufacturer's instructions. Treated cells were also stained with Oil Red O and hematoxylin as described by Suryawan and Hu [15] to visualize the lipid content. At least three images for each treatment were captured using ImagePro software (MediaCybernetics, Silver Spring, Md.).

### Cell Viability Assay.

Tests were performed in 96-well plates. Two day postconfluent preadipocytes were treated with differentiation medium containing either vehicle or test compounds for 6 days during adipogenesis. On day 6 the treatment medium was removed and the cell viability assay was performed as per the manufacturer's instructions. The absorbance was measured at 490 nm in a plate reader (μQuant Bio-Tek Instruments, Winooski, Vt.) to determine the formazan concentration, which is proportional to the number of live cells.

Apoptosis assay. For measuring the extent of apoptosis, the ApoStrand ELISA Apoptosis Detection Kit (Biomol, Plymouth Meeting, Pa.) was used. Cells were grown in 96-well plates and incubated with either vehicle or test reagents for the indicated time periods. Cells were then fixed and assayed as per the manufacturer's instructions. The assay selectively detects single-stranded DNA, which occurs in apoptotic cells but not in necrotic cells or cells with DNA breaks in the absence of apoptosis [16].

### Western Blot Analysis.

Maturing 3T3-L1 preadipocytes were treated with either carrier or test compounds (D0.5, GS3.12, and D0.5+GS3.12) from days 0-6 and whole cell extracts were prepared as described elsewhere [17]. The protein concentration was determined by BCA assay with bovine serum albumin as the standard. Western blot analysis was performed using the commercial NUPAGE system (Novex/Invitrogen), in which a lithium dodecyl sulfate sample buffer (Tris/glycerol buffer, pH 8.5) was mixed with fresh dithiothreitol and added to samples. Samples were then heated to 70° C. for 10 min, separated by 12% acrylamide gels and analyzed by immunoblotting.

### Quantitative Analysis of Western Blot Data.

Measurement of signal intensity on PVDF membranes after Western blotting with various antibodies was performed using a Fluor Chem densitomer with the Alpha-EaseFC image processing and analysis software (Alpha Innotech Corp.). For statistical analysis, all data were expressed as integrated density values (IDV), which were calculated as the density values of the specific protein bands/b-actin density values and expressed as percentage of the control. All figures showing quantitative analysis include data from at least three independent experiments.

### Statistical Analysis.

ANOVA (GLM procedure, Statistica, version 6.1; StatSoft) was used to determine significance of time and treatment effects and time vs treatment interactions. Fisher's post hoc least significant difference test was used to determine significance of differences among means. In some cases in order to estimate differences between the combined treatments and a hypothetical additive treatment response, a sum of the individual treatment effects for each replicate was calculated and these numbers were included in the ANOVA. Statistically significant differences are defined at the 95% confidence interval. Data shown are means±standard error.

### Results

### Effect of GS and 1,25(OH)2D3 on Lipid Content

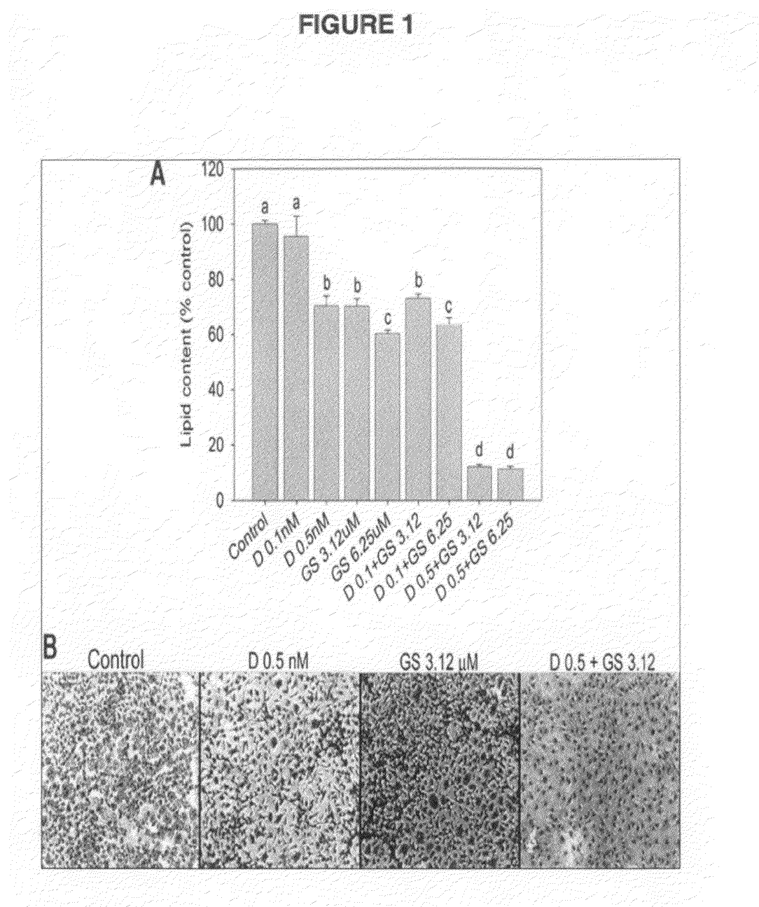

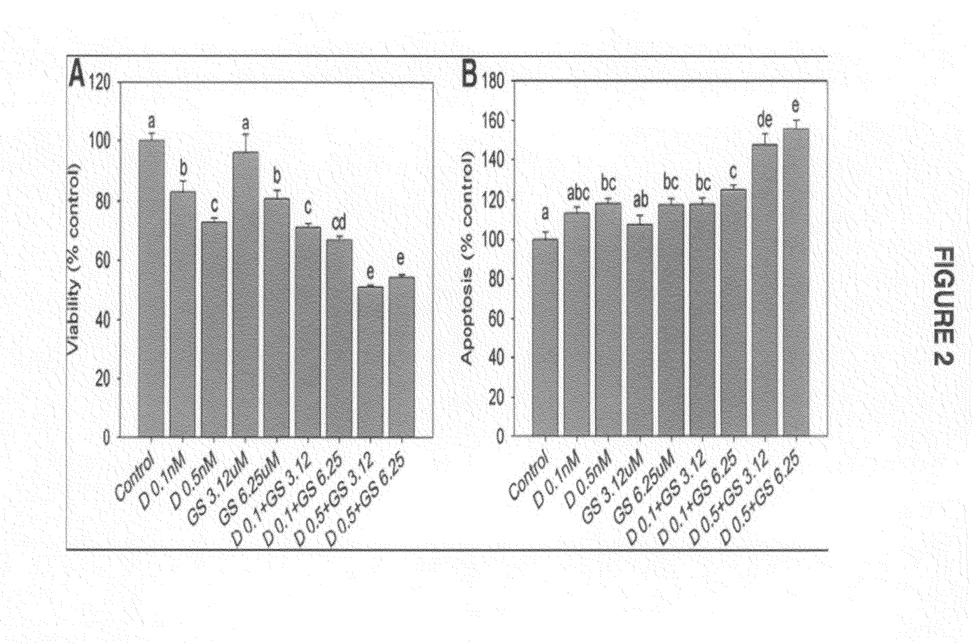

The concentration of 0.5 nM 1,25(OH)2D3 (D0.5) and 3.12 μM GS (GS3.12) as individual treatments decreased lipid accumulation by 29.3±3.4% (p<0.001) and 29.7±2.7% (p<0.001), respectively (FIG. 1A). However, the decrease in lipid accumulation caused by the D0.5+GS3.12 combination was 88.1±0.8% (p<0.001), whereas the calculated additive response of D0.5+GS3.12 would have been a decrease in lipid accumulation of 58.7±2.7%. Similar results were observed using Oil Red O staining to visualize lipid accumulation in cells after treatments (FIG. 1B). D0.5 and GS3.12 were selected for subsequent Western blotting experiments.

### Effect of GS and 1,25(OH)2D3 on Maturing Preadipocyte Viability and Apoptosis

Cell viability was decreased by 1,25(OH)2D3 alone by 26.8±1.3% (p<0.001) at 0.5 nM (D0.5); whereas, GS at 3.12 μM did not have any significant effect on cell viability. The combination of 1,25(OH)2D3 and GS (D0.5+GS3.12), however, decreased cell viability by 48.6±0.6% (p<0.001), whereas the percentage decrease in viability based on the calculated additive effect would have been 33.8±2.03% (p<0.001) (FIG. 2A). Similarly, D0.5 by itself increased apoptosis by 18.4±2.3% (p<0.05), whereas GS3.12 did not have any significant effect on apoptosis. The combination of 1,25(OH)2D3 and GS (D0.5+GS3.12) increased apoptosis by 47.1±5.8% (p<0.001), whereas the percentage increase in apoptosis based on the calculated additive effect would have been 26.3±4.4% (p<0.05) (FIG. 2B).

### Effect of GS and 1,25(OH)2D3 on PPARc, C/EBPa, and aP2 Expression

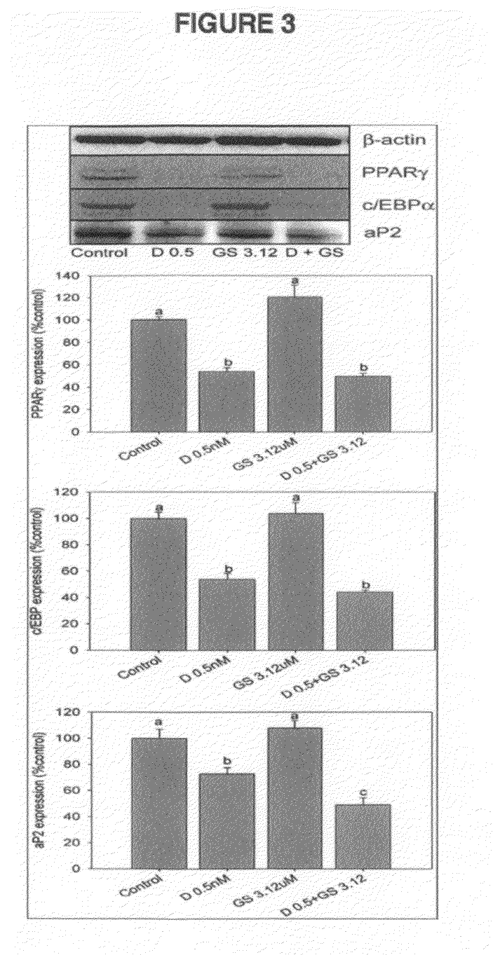

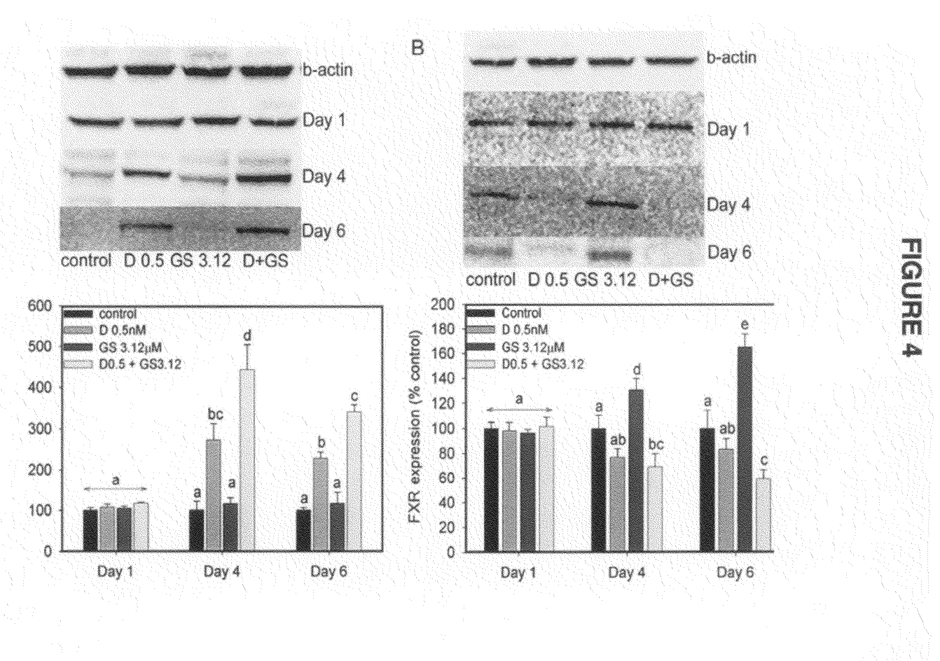

Quantitative analysis revealed that 1,25(OH)2D3 alone at the 0.5 nM concentration significantly decreased the expression of PPARc, C/EBPa, and aP2 by 46.2±4.4%, 46.3±3.4%, and 27.2±4.8% (p<0.001), respectively (FIG. 3). The treatment GS3.12, however, did not significantly alter the expression of PPARγ, C/EBPα or aP2. The combined treatment D0.5+GS3.12 decreased the expression of PPARγ and C/EBPα by 55.7±1.4% and 50.5±2.3% (p<0.001), respectively, which was not significantly different from the effect observed with D0.5 alone. However, D0.5+GS3.12 decreased aP2 expression by 50.8±5.3% (p<0.001), which is significantly different from the effect observed with D0.5 alone (p<0.05) and is also significantly different from the calculated additive effect of D0.5 and GS3.12 (FIG. 3). Effect of GS and 1,25(OH)2D3 on VDR and FXR expression Quantitative analysis revealed that 1,25(OH)2D3 alone at the 0.5 nM concentration significantly increased VDR expression levels by 172.3±40% and 128.5±40% after day 4 and day 6 (p<0.001) and did not have any significant effect on day 1. The treatment GS3.12 did not significantly alter the expression of VDR at any time point. The combined treatment D0.5+GS3.12, however, increased the expression levels of VDR by 342±61.5% and 241±16% (p<0.0001) by day 4 and day 6, respectively, while the combination had no effect on Day 1 (FIG. 4A). In contrast to the effects on VDR expression, 1,25(OH)2D3 at the 0.5 nM concentration had no effect on FXR levels at any time point, while GS at 3.12 μM concentration increased FXR levels by 30.6±9.3% and 64.8±10% on day 4 and day 6, respectively, (p<0.001) and did not have any significant effect on day 1. The treatment D0.5+GS3.12, however, decreased the expression of FXR by 30.7±10% (p=0.02) and 40.4±7.6% (p<0.05) by day 4 and day 6, respectively, while the combination had no effect on day 1 (FIG. 4B).

### Discussion

Adipocyte differentiation has been reported to be inhibited by 1,25(OH)2D3 and the prohormone also exerts antiproliferative effects in adipocytes [3,4]. GS has also been reported to induce apoptosis in cancer cells [18] and inhibit differentiation in 3T3-L1 cells [12]. In this study, we investigated the molecular events leading to the blockade of adipogenesis and induction of apoptosis in maturing 3T3-L1 preadipocytes with combined treatment of 1,25(OH)2D3 and GS. We report that the enhanced effects of 1,25(OH)2D3 plus GS on inhibition of adipogenesis and induction of apoptosis are at least partly mediated through VDR, FXR and other adipocyte-specific genes.

Inhibition of 3T3-L1 differentiation by 1,25(OH)2D3 was shown to be the result of the inhibition of glycerophosphate dehydrogenase activity and triglyceride content, counteracting the stimulatory effect of a PPARc ligand on 3T3-L1 differentiation, suppressing C/EBPa and PPARc expression and stabilizing the VDR protein [3,6,19]. Our results also revealed that 1,25(OH)2D3 at the 0.5 nM concentration significantly decreased the expression levels of C/EBPa and PPARc by 46% each. These results are in parallel with the inhibition of lipid accumulation with 1,25(OH)2D3 at the same concentration. The inhibitory actions of GS on adipocyte differentiation are mediated through inhibition of FXR [12]. Consistently, GS decreased lipid content in maturing 3T3-L1 adipocytes, but did not significantly alter the expression levels of PPARc and C/EBPa. In addition, in radioligand binding assays GS did not interact with PPARs [20]. GS and 1,25(OH)2D3 in combination, however, decreased lipid accumulation by 88%, whereas the effect of the combination on decreasing PPARc and C/EBPa expression was not significantly different from that of 1,25(OH)2D3 alone. Apart from suppressing PPARc expression, 1,25(OH)2D3 also antagonized PPARc activity [3] resulting in an enhanced decrease in lipid accumulation. This suggests that the suppression of lipid accumulation was at least partly due to antagonism of PPARc activity, which we did not directly measure, rather than to decreased PPARc expression. C/EBPa and PPARc, however, were shown to synergistically transactivate the downstream adipocyte-specific gene aP2 [21], and the combination of 1,25(OH)2D3 and GS decreased the expression of aP2 more than either compound alone, which correlates with the enhanced inhibition of lipid accumulation. Further, the decrease in lipid content might be at least in part mediated by a decrease in cell number resulting from cell death by apoptosis and probably also by inhibition of cell division during the early stage of maturation.

Previous studies from our laboratory have reported that GS induced apoptosis in mature 3T3-L1 adipocytes [22], but this is the first report of GS-induced apoptosis in 3T3-L1 maturing preadipocytes. GS-induced apoptosis was associated with induction of pro-apoptotic Bcl-2 family members like Bax and Bak in PC-3 human prostate cancer cells [23]. In mature adipocytes, GS-induced apoptosis was associated with increased caspase-3 activity and cytochrome c release from mitochondria to cytosol [22]. The antiproliferative actions of 1,25(OH)2D3 were mediated through VDR, which is expressed at high levels early in adipogenesis [6]. In agreement with the previous findings [4], 1,25(OH)2D3 treatment induced apoptosis in maturing 3T3-L1 adipocytes. Interestingly, 1,25(OH)2D3 and GS in combination led to a potentiated increase in apoptosis.

FXR is a member of the nuclear hormone receptor superfamily that was identified as the physiological receptor for bile acid [24]. Studies show that FXR is not expressed in 3T3-L1 preadipocytes, but the FXR mRNA levels are robustly increased with the induction of differentiation [12]. Exposure of 3T3-L1 cells to potent and selective FXR ligands increases preadipocyte differentiation, and GS, which is a known FXR antagonist [25], reversed this effect [12]. In this study GS increased FXR expression in maturing preadipocytes time dependently, with the expression being highest on day 6. The combination of 1,25(OH)2D3 and GS, however, significantly decreased FXR levels. We propose that GS is acting like an inverse agonist at lower concentrations which explains the upregulation of FXR levels upon GS treatment. Further, induction of apoptosis by GS in a Barrett's esophagus-derived cell line suggests that FXR may contribute to the regulation of apoptosis [26]. This is the first study to report that GS at lower concentrations increased the expression of FXR levels in 3T3-L1 adipocytes. VDR protein levels drastically increase after the induction of differentiation and, in contrast to FXR, gradually decline during the progression of the differentiation process [6]. The treatment of the 3T3 cells with 1,25(OH)2D3 stabilizes VDR levels to exert antiproliferative effects and inhibit adipogenesis [3,6]. In the present study, 1,25(OH)2D3 alone increased VDR levels by 170% by day 4 and 130% by day 6. GS at 3.12 μM, however, did not significantly alter VDR levels. The combination upregulated VDR expression significantly more than 1,25(OH)2D3 alone on both days 4 and 6. Even though GS was reported to be a promiscuous steroid receptor ligand [20], the effect on or affinity of GS for VDR has not previously been investigated. VDR and 1,25(OH)2D3 were reported to have a profound effect on the signal transduction mediated by bile acid/FXR [13].

Further, VDR suppressed the transactivation driven by bile acid/FXR in a 1,25(OH)2D3-dependent manner [13]. Interestingly, a significant increase in VDR levels by 1,25(OH)2D3 and GS combination treatment on days 4 and 6 was associated with a decreasing trend in FXR levels, which may suggest that VDR activation by 1,25(OH)2D3 reduces expression of FXR. Nuclear receptor signaling pathways include stimulus (ligand), ligand/receptor interaction, dimerization, coreceptor activation, and finally increased transcription of a battery of target genes. Crosstalk among nuclear receptors, like liver X receptors, thyroid hormone receptors, pregnane X receptors, including FXR and VDR, can result at any one of these steps [27]. In the current study, treatment with 1,25(OH)2D3 and GS resulted in increased VDR expression levels in parallel with decreased FXR levels, indicating a possible crosstalk between these two nuclear receptors. In conclusion, we demonstrated that in 3T3-L1 maturing preadipocytes 1,25(OH)2D3 and GS at tested concentrations had little or no effect as individual treatments, but in combination they were more potent in inducing apoptosis and decreasing lipid accumulation, and thus may be acting in a synergistic fashion.

### Further Examples

### Enhanced Osteogenesis with 1,25(OH)₂D₃+Guggulsterone and 1,25(OH)₂D₃+Xanthohumol

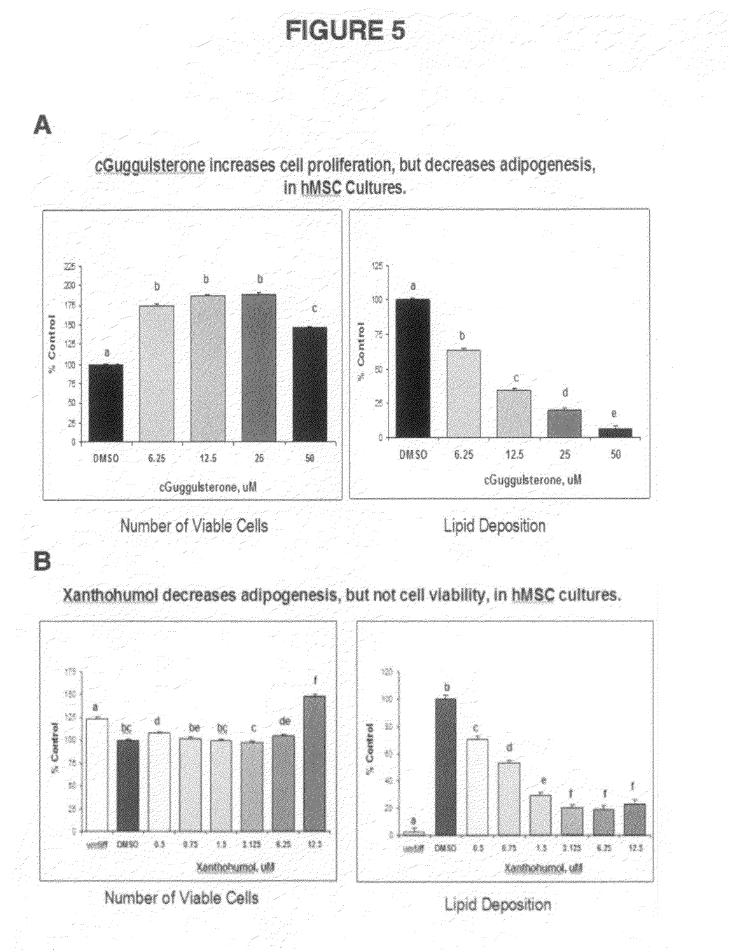

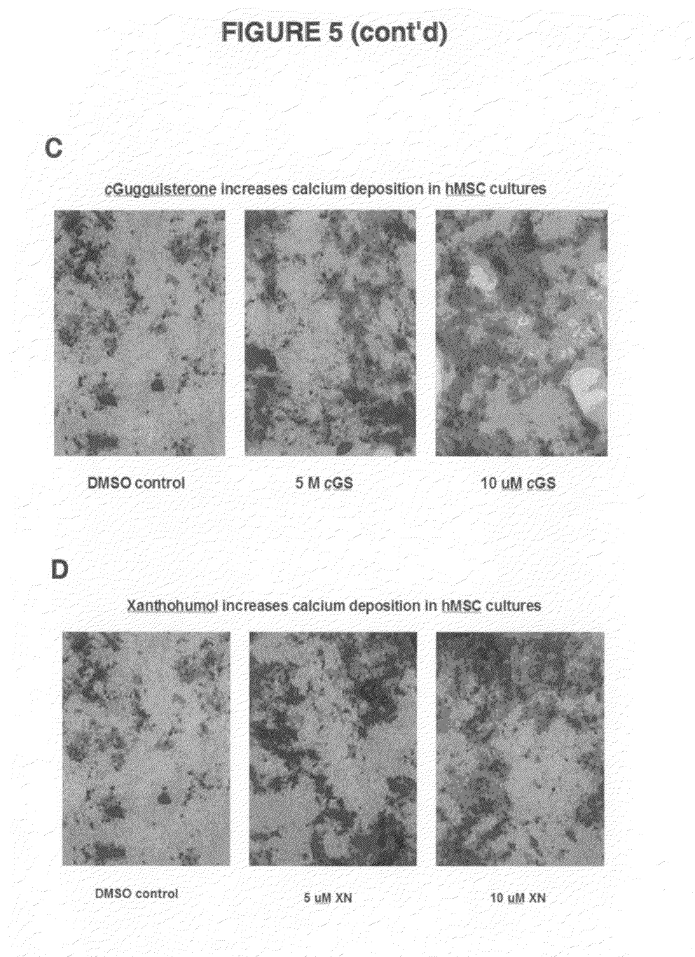

The above examples evidenced that both cis-guggulsterone (cGS) and xanthohumol (XN) enhanced osteogenesis and suppressed adipogenesis in human mesenchymal stem cells (hMSC), cells that are the precursors of adipocytes and bone forming cells (osteoblasts) in bone marrow. In these further examples, post confluent hMSC were treated with varying doses of cGS or XN in adipogenic induction medium, and lipid deposition and cell viability were measured. Alternatively, pre-confluent hMSC were pre-treated with varying doses of cGS or XN for 3 days prior to addition of osteogenic induction medium. Alkaline phosphatase (ALP) activity was determined 3 days post-induction and indices of mineralization (calcium deposition and Alizarin red S stain) were determined 14-28 days post-induction. Results (FIG. 5A-D) indicated dose-dependent increase in cell number (viability) after cGS treatment under either adipogenic or osteogenic conditions, whereas XN increased cell viability dose-dependently (1.5-12 μM; P<0.05) in hMSC adipogenic cultures but decreased viability at high levels (20 μM) in osteogenic cultures. Both compounds inhibited lipid deposition in hMSC cultured under adipogenic conditions. Inhibition was dose-dependent with ˜50% inhibition observed with 6.5 μM cGS and 0.75 μM XN, respectively. Pretreatment with cGS stimulated osteogenic differentiation in hMSC cultures as indicated by increased ALP activity at day 3 (˜10%; P<0.05) and calcium deposition at day 17 (˜35-60%; P<0.05). Pretreatment with 5 μM XN increased calcium deposition (33%; P<0.05) at day 17.

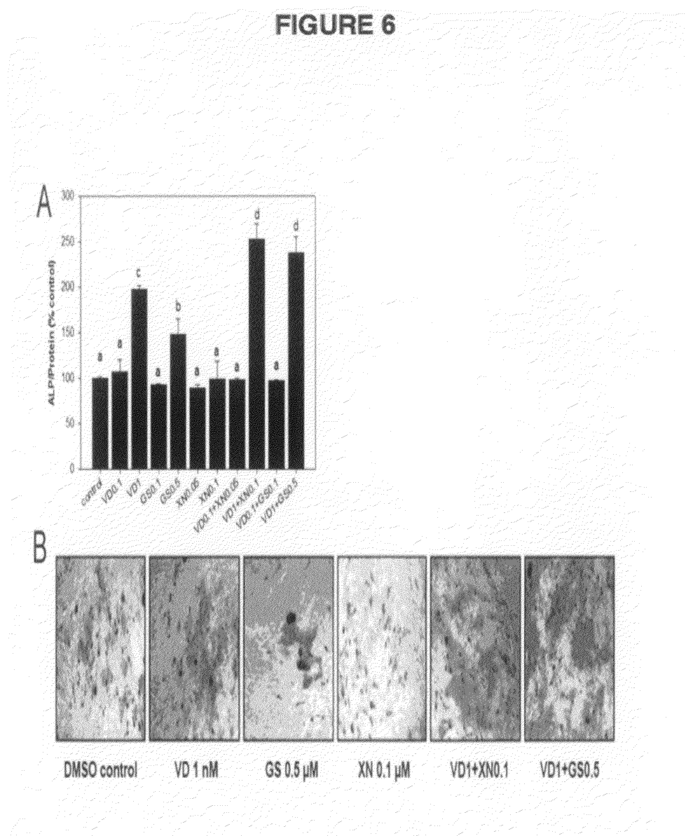

In similar experiments testing the effects of vitamin D combined with either GS or XN, both combinations were found to increase ALP activity and calcium deposition more than vitamin D alone (P<0.05) (FIG. 6).

### Dietary Supplementation of Vitamin D (VD)+Genistein (G)+Resveratrol (R)+Quercetin (Q) Reduces Weight Gain and Body Fat and Increased Bone Density in Ovariectomized Female Rats

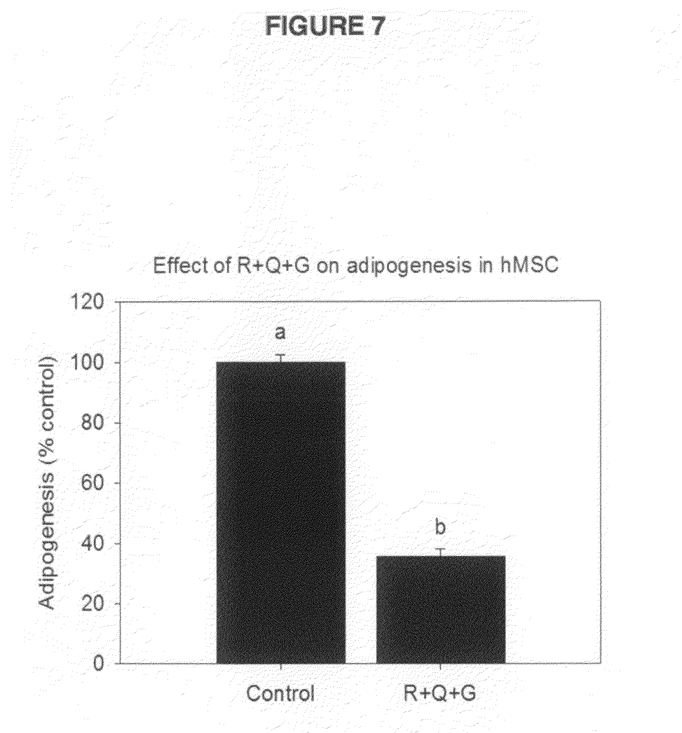

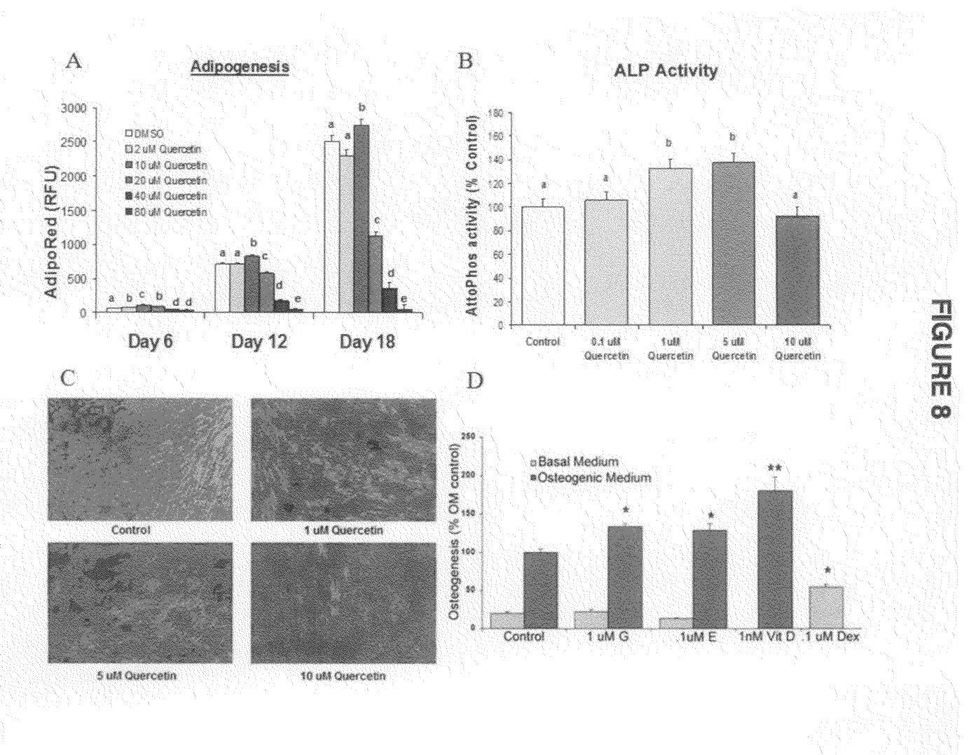

In previous in vitro experiments, the inventors found that quercetin+resveratrol+genistein suppressed adipogenesis in bone precursor cells (hMSC) (FIG. 7), and that vitamin D, quercetin and genistein promoted osteogenesis (FIG. 8).

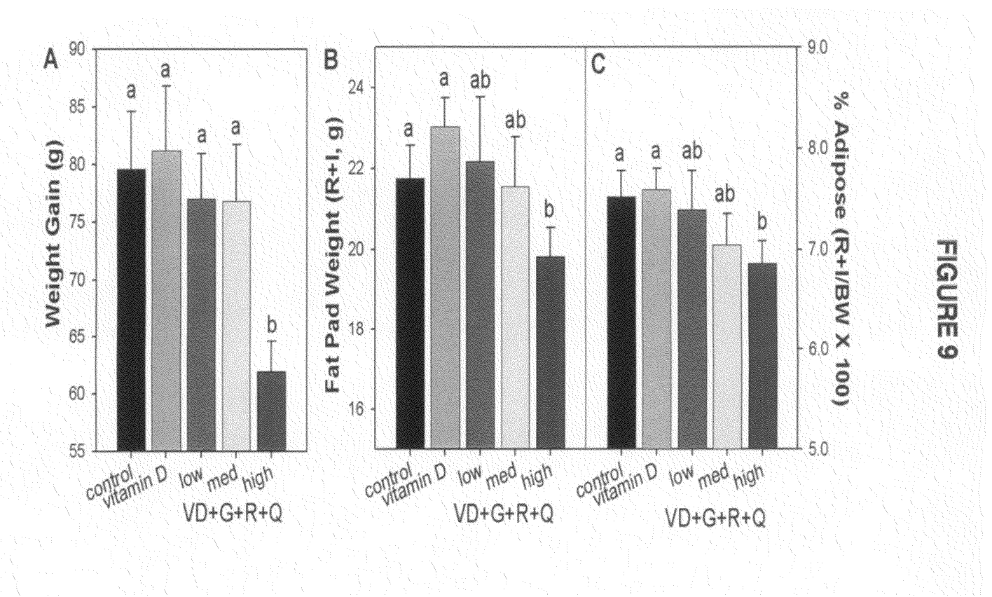

This in vivo experiment was designed to determine the effectiveness of vitamin D+R+Q+G in reducing adiposity and preventing bone loss in a rodent model of post-menopausal osteoporosis and weight gain. Twelve month old ovariectomized female rats (N=10) were treated for 8 weeks with control, vitamin D alone (0.2 mg/kg BW/d), or vitamin D+resveratrol (1, 5, or 25 mg/kg/d)+quercetin (5, 25 or 125 mg/kg/d)+genistein (4, 16 or 65 mg/kg/d). Retroperitoneal (R) and inguinal (I) fat pads were collected and weighed. Femora were collected and processed for various types of analyses. Compared to all other treatments, the high dose combination treatment significantly reduced weight gain, weight of fat pads (R+I) and R+I as % of body weight (FIG. 9).

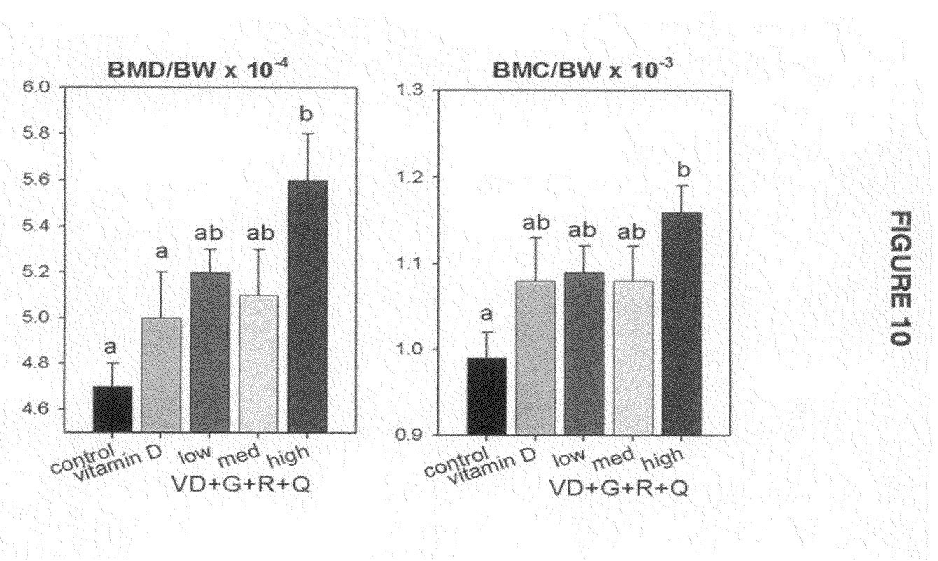

Femora were analyzed by densitometry to determine bone mineral density (BMD) and content (BMC). BMD was significantly increased by the high dose combination treatment compared to both control and vitamin D alone (FIG. 10). BMC was significantly increased by the high dose combination treatment compared to control.

The above study shows that a combination of vitamin D with natural compounds selected on the basis of activity in in vitro adipocyte and mesenchymal stem cell assays can have activity in vivo in a model of post-menopausal weight gain and bone loss. The weight adjusted improvement in bone density, along with a reduction in weight gain and adiposity is an important finding, because decreased adiposity is typically associated with decreased bone density.

### REFERENCES

[1] D. Somjen, A. M. Kaye, A. Harell, Y. Weisman, Modulation by vitamin D status of the responsiveness of rat bone to gonadal steroids, Endocrinology 125 (1989) 1870-1876.
[2] Y. S. Zhou, Y. S. Liu, J. G. Tan, Is 1,25-dihydroxyvitamin D3 an ideal substitute for dexamethasone for inducing osteogenic differentiation of human adipose tissue-derived stromal cells in vitro? Chin. Med. J. (Engl) 119 (2006) 1278-1286.
[3] J. Kong, Y. C. Li, Molecular mechanism of 1,25-dihydroxyvitamin D3 inhibition of adipogenesis in 3T3-L1 cells, Am. J. Physiol. Endocrinol. Metab. 290 (2006) E916-E924.
[4] X. Sun, M. B. Zemel, Role of uncoupling protein 2 (UCP2) expression and 1alpha, 25-dihydroxyvitamin D3 in modulating adipocyte apoptosis, FASEB J. 18 (2004) 1430-1432.
[5] T. E. Hedlund, K. A. Moffatt, G. J. Miller, Stable expression of the nuclear vitamin D receptor in the human prostatic carcinoma cell line JCA-1: evidence that the antiproliferative effects of 1 alpha, 25-dihydroxyvitamin D3 are mediated exclusively through the genomic signaling pathway, Endocrinology 137 (1996) 1554-1561.
[6] J. M. Blumberg, I. Tzameli, I. Astapova, F. S. Lam, J. S. Flier, A. N. Hollenberg, Complex role of the vitamin D receptor and its ligand in adipogenesis in 3T3-L1 cells, J. Biol. Chem. 281 (2006) 11205-11213.
[7] M. L. Drent, E. A. van der Veen, Endocrine aspects of obesity, Neth. J. Med. 47 (1995) 127-136.
[8] R. Scragg, I. Holdaway, V. Singh, P. Metcalf, J. Baker, E. Dryson, Serum 25-hydroxyvitamin D3 levels decreased in impaired glucose tolerance and diabetes mellitus, Diabetes Res. Clin. Pract. 27 (1995) 181-188.
[9] S. Shishodia, B. B. Aggarwal, Guggulsterone inhibits NF-kappaB and IkappaBalpha kinase activation, suppresses expression of anti-apoptotic gene products, and enhances apoptosis, J. Biol. Chem. 279 (2004) 47148-47158.
[10] G. V. Satyavati, Gum guggul (Commiphora mukul)—the success story of an ancient insight leading to a modern discovery, Indian J. Med. Res. 87 (1988) 327-335.
[11] N. L. Urizar, D. D. Moore, GUGULIPID: a natural cholesterol lowering agent, Annu. Rev. Nutr. 23 (2003) 303-313.
[12] G. Rizzo, M. Disante, A. Mencarelli, B. Renga, A. Gioiello, R. Pellicciari, S. Fiorucci, The farnesoid X receptor promotes adipocyte differentiation and regulates adipose cell function in vivo, Mol. Pharmacol. 70 (2006) 1164-1173.
[13] Y. Honjo, S. Sasaki, Y. Kobayashi, H. Misawa, H. Nakamura, 1,25-dihydroxyvitamin D3 and its receptor inhibit the chenodeoxycholic acid-dependent transactivation by farnesoid X receptor, J. Endocrinol. 188 (2006) 635-643.
[14] N. Hemati, S. E. Ross, R. L. Erickson, G. E. Groblewski, O. A. MacDougald, Signaling pathways through which insulin regulates CCAAT/enhancer binding protein alpha (C/EBPalpha) phosphorylation and gene expression in 3T3-L1 adipocytes. Correlation with GLUT4 gene expression, J. Biol. Chem. 272 (1997) 25913-25919.
[15] A. Suryawan, C. Y. Hu, Effect of serum on differentiation of porcine adipose stromal-vascular cells in primary culture, Comp. Biochem. Physiol. Comp. Physiol. 105 (1993) 485-492.
[16] O. S. Frankfurt, Immunoassay for single-stranded DNA in apoptotic cells, Methods Mol. Biol. 282 (2004) 85-101.
[17] J. Y. Yang, M. A. Della-Fera, C. Nelson-Dooley, C. A. Baile, Molecular mechanisms of apoptosis induced by ajoene in 3T3-L1 adipocytes, Obesity (Silver Spring) 14 (2006) 388-397.
[18] I. Samudio, M. Konopleva, S. Safe, T. McQueen, M. Andreeff, Guggulsterones induce apoptosis and differentiation in acute myeloid leukemia: identification of isomer-specific antileukemic activities of the pregnadienedione structure, Mol. Cancer. Ther. 4 (2005) 1982-1992.
[19] Y. Hida, T. Kawada, S. Kayahashi, T. Ishihara, T. Fushiki, Counteraction of retinoic acid and 1,25-dihydroxyvitamin D3 on up-regulation of adipocyte differentiation with PPAR gamma ligand, S. Rayalam et al./Biochemical and Biophysical Research Communications 364 (2007) 450-456 455

1. A composition comprising an effective amount of vitamin D3 or an analog thereof in combination with an effective amount of at least one additional compound selected from the group consisting of genistein, guggulsterone and xanthohumol or a pharmaceutically acceptable salt thereof in combination with a pharmaceutically acceptable carrier, additive or excipient.

2. A composition according to claim 1 wherein said additional compound is genistein.

3. A composition according to claim 1 wherein said additional compound is guggulsterone.

4. A composition according to claim 1 wherein said additional compound is xanthohumol.

5. A composition according to claim 1 wherein said additional compound is a mixture of two compounds.

6. The composition according to claim 5 wherein said two compounds are guggulsterone and xanthohumol.

7. The composition according to claim 5 wherein said two compounds are guggulsterone and genistein.

8. The composition according to claim 5 wherein said two compounds are xanthohumol and genistein.

9. The composition according to claim 1 wherein said additional compound is a mixture of all three compounds.

10. A composition according to claim 1 wherein vitamin D3 comprises about 25 μg to about 1.25 mg of said composition, genistein, when used, comprises about 5 mg to about 500 mg of said composition, guggulsterone, when used, comprises about 5 mg to about 500 mg of said composition and xanthohumol, when used, comprises about 500 μg to about 250 mg of said composition.

11. The composition according to claim 1 in oral dosage form.

12. The composition according to claim 1 in sublingual or buccal dosage form.

13. A composition comprising an effective amount of guggulsterone and xanthohumol optionally in combination with an effective amount of at least one additional compound selected from the group consisting of genistein and vitamin D3 or an analog thereof in combination with a pharmaceutically acceptable carrier, additive or excipient

14. The composition according to claim 13 wherein guggulsterone comprises about 5 mg to about 500 mg of said composition, xanthohumol comprises about 500 μg to about 250 mg of said composition, vitamin D3, when used, comprises about 25 μg to about 1.25 mg of said composition, and genistein, when used, comprises about 5 mg to about 500 mg of said composition.

15. The composition according to claim 13 adapted for oral administration.

16. The composition according to claim 13 adapted for sublingual or buccal administration.

17. The composition according to claim 1 wherein said vitamin D3 analog is 1,25 dihydroxy vitamin D3 (calcitriol) or cholecalciferol.

18. The composition according to claim 17 wherein said vitamin D3 analog is 1,25 dihydroxy vitamin D3 (calcitriol).

19. The composition according to claim 17 wherein said vitamin D3 analog is cholecalciferol.

20. The composition according to claim 1 wherein said carrier is a polymeric material which slowly releases said composition at a site of a bone graft.

21. The composition according to claim 20 wherein said polymeric material is a collagen matrix.

22. The composition according to claim 1 further comprising an effective amount of quercetin, resveratrol or mixtures thereof.

23. A method of treating osteoporosis in a patient in need thereof comprising administering an effective amount of a composition according to claim 1 to said patient.

24. A method of treating obesity in a patient in need thereof comprising administering an effective amount of a composition according to claim 1 to said patient.

25. A method of reducing body fat in a patient comprising administering an effective amount of a composition according to claim 1 to said patient.

26. The method according to claim 25 wherein said body fat is visceral body fat.

27. A method of enhancing a bone graft in a patient said method comprising administering to a site of a bone graft in said patient an effective amount of a composition according to claim 1.

28. (canceled)

29. A method of enhancing osteogenesis in a patient comprising administering to said patient an effective amount of a composition according to claim 1.

30. A method of simultaneously reducing body fat and enhancing osteogenesis in a patient comprising administering to said patient an effective amount of a composition according to claim 1.
